(19) United States
(12) Plant Patent Application Publication
(10) Pub. No.: US 2002/0184686 P1
(43) Pub. Date: Dec. 5, 2002

(54) Chrysanthemum plant named 'Gedi One Ibe'

(76) Inventor: Dirk Pieters, Staden (BE)

Correspondence Address: KNOBBE MARTENS OLSON & BEAR LLP, 2040 MAIN STREET, FOURTEENTH FLOOR, IRVINE, CA, 92614, US

(21) Appl. No.: 09/873,876

(22) Filed: Jun. 4, 2001

(51) Int. Cl. A01H 5/00

(52) U.S. Cl. PLT/294

(57) ABSTRACT

A new and distinct Chrysanthemum plant variety characterized by a decorative type inflorescence, consistent flowering response to short days, blooming consistently after 49 days of short day length, free branching habit, white ray florets, very uniform round growth habit, and a large quantity of blooms per flowering branch.

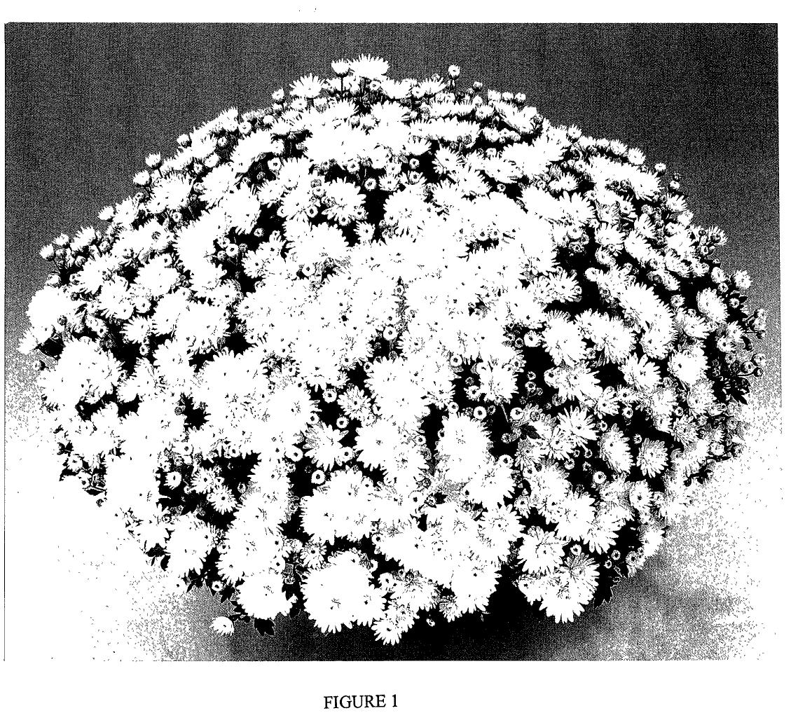

### BACKGROUND OF THE INVENTION

The present invention relates to a new and distinct cultivar of Chrysanthemum plant, botanically known as Dendranthema morifolium and herein after referred to by the cultivar name ‘GEDI ONE IBE.’

This new cultivar originated from hybridization of a female parent, cultivar ‘Cassablanco’ (unpatented in the United States), and a male parent cultivar ‘Papiro’ (U.S. patent Ser. No. 09/550,157). The new cultivar was discovered by the inventor in October 1997.

Asexual reproduction of the new cultivar by apical tip cutting and meristem tissue culture was performed in Oxnard, Calif. and has shown that the unique features of this new cultivar are stable and reproduced true to type on successive generations.

### SUMMARY OF THE INVENTION

The cultivar ‘GEDI ONE IBE’ has not been observed under all possible environmental conditions. The phenotype may vary somewhat with variations in environment such as temperature, daylength, and light intensity, without, however, any variance in genotype.

The following traits have been repeatedly observed and are determined to be the unique characteristics of ‘GEDI ONE IBE.’ These characteristics in combination distinguish ‘GEDI ONE IBE’ as a new and distinct Chrysanthemum:

1. Decorative type inflorescence;

2. Consistent flowering response to short days, blooming consistently after 49 days of short day length;

3. Free branching habit;

4. White ray florets;

5. Very uniform round growth habit; and

6. Large quantity of blooms per flowering branch.

Plants of the new cultivar are similar to plants of the parent cultivar, ‘Cassablanco,’ in most horticultural characteristics, however plants of the new cultivar differed from plants of the cultivar ‘Cassablanco’ in the following characteristics:

1. The new cultivar has more blooms per flowering branch;

2. The new cultivar has many more ray florets; and

3. The new cultivar has a wider diameter bloom.

Plants of the new cultivar are also similar to plants of the parent cultivar, ‘Papiro,’ in most horticultural characteristics, however plants of the new cultivar differed from plants of the cultivar ‘Papiro’ in the following characteristics:

1. The new cultivar has a different flower type;

2. The new cultivar is taller and wider; and

3. The new cultivar has a wider diameter bloom.

The new cultivar can be compared to the Chrysanthemum cultivar ‘Odysseus’ (disclosed in U.S. Plant Pat. No. 11,637). However plants of the new Chrysanthemum differed from the plants of the cultivar ‘Odysseus’ in the following characteristics:

1. The new cultivar naturally blooms 2 weeks later,

2. The new cultivar has a more flexible plant habit;

3. The new cultivar has a more round plant shape and greater branching; and

4. The new cultivar has more blooms per flowering branch.

### BRIEF DESCRIPTION OF THE PHOTOGRAPHS

The photograph shows a plant of ‘GEDI ONE IBE’ grown in a 2-gallon container. A single cutting was used in the pot, planted mid-May and grown under outdoor field conditions.

### DETAILED BOTANICAL DESCRIPTION

In the following description, color references are made to The Royal Horticultural Society Colour Chart except where general terms of ordinary dictionary significance are used. The following observations and measurements describe plants grown in a commercial filed on Oxnard, Calif. in the month of September 2000. The growing conditions range from 60° to 65° F. at night to 70° to 85° during the day. Measurements and numerical values represent averages of typical flowering types.

Botanical classification: Dendranthema morifolium cultivar ‘GEDI ONE IBE’

Commercial classification: Garden-type Chrysanthemum

Parentage: Hybridization of female parent ‘Cassablanco’ (unpatented in the United States) and male parent ‘Papiro’ (U.S. patent Ser. No. 09/550,157)

### Propagation

Time to Rooting: 7 to 14 days at approximately 21° C.

Rooting habit: Fine, fibrous

### Plant

Growth Habit: Mounding herbaceous perennial

Height: Approximately 29 cm

Growth rate: Moderate

Branching Characteristics: Free Branching

Length of Lateral Branches: Approximately 24 cm

Number of leaves per lateral branch: Approximately 23

Stem color: Near 137A

### Foliage

Leaf:

Arrangement.—Alternate single

Average Length.—Approximately 7.1 cm

Average Width.—Approximately 4.2 cm

Shape of blade.—Ovate

Apex.—Acuminate

Base.—Attenuate

Attachment.—Stalked

Margin.—Serrate

Texture of top surface.—Lightly pubescent

Texture of bottom surface.—Lightly pubescent

Color: young foliage upper side: Near 137A young foliage under side: Near 137A mature foliage upper side: Near 137A mature foliage under side: Near 137A venation upper side: Near 137C venation underside: Near 137C

Petiole:

Average Length.—Approximately 2.5 cm

### Bloom

Inflorescence:

Flowering habit.—Induced by darkness period greater than 13.5 hours, approximately 49 days of appropriate day length required to induce and develop blooms

Inflorescence form.—Decorative

Natural flowering season.—Approximately the last two weeks of August

Number of inflorescences per stem.—Approximately 13

Inflorescence diameter.—Approximately 7.5 cm

Inflorescence depth.—2.0 cm

Inflorescence longevity on plant.—Approximately 22-25 days

Persistence.—Persistent

Ray Florets:

Appearance.—Matte

Texture.—Smooth

Average number per flower.—Approximately 300

Shape.—Oblanceolate

Margin.—Entire

Apex.—Obtuse

Length.—Approximately 4.2 cm

Width.—Approximately 1.0 cm

Color.—upper surface at first opening: Near 13D upper surface at maturity: Near 155D upper surface at fading: Near 155D under surface at first opening: Near 18B under surface at maturity: Near 11D under surface at fading: Near 155D

Disc Florets:

Appearance.—Shiny

Texture.—Smooth

Average numberperflower.—Approximately 40

Shape.—Cylindric

Apex.—Obtuse

Average Length.—Approximately 0.8 cm

Average Width.—Approximately 0.2 cm

Color.—at first opening: Near 3D at maturity: Near 3B at fading: Near 16C

Peduncle:

Length.—at terminal end (shortest): Approximately 6 cm at lateral end (longest): Approximately 9 cm

Strength.—Moderate

Color.—Near 137C

Habit.—Erect

Inflorescence Bud:

Length.—Approximately 1.3 cm

Diameter.—Approximately 1.2 cm

Form.—Globular

Color.—Near 18D

Sepals:

Appearance.—Matte

Texture.—Lightly pubescent

Number.—Approximately 50 to 60

Shape.—Ovate

Margin.—Entire

Apex.—Acute

Color.—upper side: Near 137B under side: Near 137B

### Reproductive Organs

Ray Florets:

Pistillate.—Number of pistils perflower: 1 Stigma shape: 2 branched Stigma color: Near 10B Style color: Near 10D Style length: Approximately 0.7 cm

Disc Florets:

Pistillate.—Number of pistils per flower: 1 Stigma shape: Cylindric Style length: Approximately 0.5 cm

Staminate.—Number of stamens per flower: Approximately 5 Anther shape: Tubular Anther color: Near 18A Pollen color: Near 17C

### Other Characteristics

Disease Resistance: Neither resistance nor susceptibility observed

What is claimed is:

1. A new and distinct variety of Chrysanthemum plant named ‘GEDI ONE IBE’ as herein illustrated and described.
